(19) United States
(12) Patent Application Publication
(10) Pub. No.: US 2025/0049084 A1
(43) Pub. Date: Feb. 13, 2025

(54) PROCESS FOR PREPARING A BERRY COMPOSITION

(71) Applicant: SOCIETE DES PRODUITS NESTLE S.A., Vevey (CH)

(72) Inventors: YONGCHENG LIAO, Beijing (CN); HUAN SHI, Beijing (CN); CHUNHUA DONG, Beijing (CN); LENNART FRIES, Beijing (CN); FEI XU, Lausanne (CH); YUE SONG, Shandong (CN)

(21) Appl. No.: 18/720,702

(22) PCT Filed: Dec. 19, 2022
(86) PCT No.: PCT/CN2022/139992

§ 371 Date: Jun. 17, 2024

(30) Foreign Application Priority Data
Dec. 20, 2021 (WO) PCT/CN2021/139761

(51) Int. Cl. A23L 19/00; A23L 33/19

(52) U.S. Cl. CPC A23L 19/09; A23L 33/19

(57) ABSTRACT

The invention relates to a process for preparing a berry composition from whole berries having bioactive and/or nutritional compounds of a whole berry and having improved sensory and miscibility/dispersibility properties. The process comprises the steps of providing whole berries, grinding said whole berries to obtain a whole berry suspension and micronizing said whole berry suspension. In particular, the process does not comprise any step of removal of the inherent insoluble fibers coming from the whole berries. The invention also relates to a berry composition, to oral compositions comprising such as berry composition and to the use of such compositions.

### TECHNICAL FIELD

The present invention relates generally to the field of processes for preparing a berry composition, preferably wolfberry composition, comprising all essential bioactive and/or nutritional compounds of a whole berry and having at least improved sensory and miscibility/dispersibility properties. It also relates to such a berry composition, to an oral composition comprising such a berry composition and to the use of such compositions.

### BACKGROUND OF THE INVENTION

Pigments, bioactive and/or nutritional compounds extracted from fruits, in particular berries, are widely used in the food industry as functional ingredients. Among all of them, Lycium barbarum and Lycium Chinense are one of the most valued functional ingredients in China, especially for their benefits for eyesight, the immune system, and its anti-ageing properties, associated with the multiple bioactive and/or nutritional compounds present in their fruits, named wolfberries or Lycii fructus. It is traditionally consumed through hot water extraction.

Many other ingredients are also well perceived by consumers for their beneficial properties, but their applications in food products are either difficult or give poor bioavailability. In fact, fruits are usually rich in reducing sugars, making the drying and handling of their powder very difficult.

A large number of extraction techniques are already known and generally involve solvents. For example, in WO03020053, a process for extracting carotenoids from carotenoid-containing plant matter is described. It comprises (i) mixing the plant matter with water to achieve Brix not greater than 10° C.; (ii) crushing the mixture from stage (i) and separating the solids from the liquid to obtain two phases, pulp and serum; and (iii) extracting the pulp with solvents, e.g., ethyl acetate, to obtain carotenoid-containing plant oleoresin.

For example, U.S. Pat. No. 6,648,564 describes a process for forming, isolating and purifying xanthophyll crystals by saponification of a xanthophyll diester-containing plant extract in a composition of propylene glycol and aqueous alkali to form xanthophyll crystals. The substantially pure xanthophyll crystals so obtained are suitable for human consumption and can be used as a nutritional supplement and as an additive in food.

However, solvent extraction techniques are more difficult to handle, and using solvent can impair the natural image and/or nutritional functions of the product.

Moreover, conventional extraction techniques usually extract a few compounds of the fruit material, leaving some other bioactive compounds in the rest. For example, polysaccharides, polyphenols and other non-lipophilic compounds are not extracted together with the lipophilic components such as carotenoids, lipophilic vitamins and other lipids.

For example, U.S. Pat. No. 6,409,996 B1 describes a method of obtaining a composition comprising one or more flavonoids by treating a flavonoid containing raw material with an aqueous extraction medium to obtain an extract and separating the flavonoids from said extract by absorption and/or adsorption. Such extraction method only gives an extract mainly containing a part of bioactive principles of the raw material.

Moreover, there have been some attempts to extract bioactive and/or nutritional compounds together from plant or fruit materials. However, the insoluble and/or solid materials, mainly coming from skin, pulp and seeds, are discarded during extraction, for example by centrifugation, decanting, filtration. This exclusion of insoluble and/or solid materials ensures a good miscibility and maintains an acceptable sensory performance (e.g. without perception of coarse particle in mouth) of the resulting extracted composition. However, this limits the yield of extraction as part of the bioactive and/or nutritional compounds of interest, including soluble compounds, are discarded with the solid and insoluble materials. In addition, insoluble compounds which have key nutritional and health-related properties are lost in majority when discarding the solid and/or insoluble materials. For example, a major part of fibers, including insoluble dietary fibers, which are key nutritional and health-related compounds are lost when discarding the solid and/or insoluble materials. Moreover, this exclusion of the insoluble and/or solid materials generate waste materials which are generally lost.

For example, WO200592121 A1 describes an extraction process for obtaining a primary composition comprising the essential lipophilic and hydrophilic bioactive components of a fruit, vegetable and/or plant material in a milk or milk protein-containing carrier. The solid and insoluble materials, in particular insoluble fibers, are excluded. In particular, around 30-40% compounds of the fruit, vegetable and/or plant materials are lost during processing and should be treated as a waste. This limits significatively the extraction yield and retention rate of the bioactive and/or nutritional compounds from the whole fruit, vegetable and/or plant materials.

It would therefore be desirable to address the above problems. In particular, it would be desirable to provide a process for preparing a composition by extracting the multi-nutrients, in particular the different bioactive and/or nutritional compounds, including insoluble fibers, from whole berries, preferably whole wolfberries, with an improved extraction/retention yield and with no or at least limited waste. It would be desirable that the process provides a berry composition, prepared from whole berries, with improved sensory properties, miscibility/dispersibility properties in aqueous systems, and preferably with enhanced stability and bioavailability of the bioactive and/or nutritional compounds.

It would also be desirable to provide a berry composition that can be used directly or easily concentrated or dried into powder for the formulation of food, beverage, food supplement, pharmaceutical or cosmetic oral composition.

It is also desirable to provide a composition having the different bioactive and/or nutritional compounds from whole berries, preferably whole wolfberries, such that the composition can be used for different cosmetic and/or pharmaceutical applications.

Any reference to prior art documents in this specification is not to be considered an admission that such prior art is widely known or forms part of the common general knowledge in the field.

### SUMMARY OF THE INVENTION

The object of the present invention is to improve the state of the art, and in particular to provide a process, compositions, method and uses that overcome the problems of the prior art and addresses the needs described above, or at least to provide a useful alternative.

It has been surprisingly found that the object of the present invention could be achieved by the subject matter of the independent claims. The dependent claims further develop the idea of the present invention.

Accordingly, a first aspect of the invention proposes a process for preparing a berry composition comprising the steps consisting of:
i) providing whole berries, wherein the whole berries comprise skin, seeds and/or pulp which are intact, and wherein the whole berries comprise inherent insoluble fibers,
ii) grinding the whole berries to prepare a berry suspension,
iii) micronizing said whole berries in presence of a liquid medium to prepare a micronized berry suspension having D(90) particle size of less than 200 μm,
iv) optionally, mixing and/or milling the micronized berry suspension in milk or milk protein-containing liquid medium to obtain a milk protein-containing micronized berry suspension,
v) optionally heat-treating the micronized berry suspension or the milk protein-containing micronized berry suspension,
vi) optionally add synthetic or natural bioactive ingredients to the micronized berry suspension or the milk protein-containing micronized berry suspension,
vii) optionally drying the micronized berry suspension or the milk protein-containing micronized berry suspension to obtain a powder,
wherein the process does not comprise any step of removal of the inherent insoluble fibers coming from the whole berries.

This process enables to provide a berry composition comprising the multi-nutrients, including insoluble fibers, of whole berries with an improved extraction/retention yield and with no or at least limited waste by retaining the compounds present in the insoluble and/or solid materials which are generally discarded in the art. In particular, the process enables to provide a berry composition with improved sensory properties, miscibility/dispersibility properties in aqueous systems, and preferably with enhanced stability and bioavailability of the bioactive and/or nutritional compounds while retaining the compounds from the insoluble and/or solid materials.

A second aspect of the invention proposes a berry composition which comprises lipophilic and hydrophilic, bioactive and/or nutritional compounds of a whole berry, which comprises the soluble and insoluble fibers of a whole berry, which has a D(90) particle size of less than 200 μm and which has at least 1.5 wt. % of soluble and insoluble fibers.

The berry composition has a profile of bioactive and/or nutritional compounds which is close to that naturally occurred in whole berries while having improved sensory properties, miscibility/dispersibility properties in aqueous systems, and preferably with enhanced stability and bioavailability of the bioactive and/or nutritional compounds. In particular, it has a substantial amount of insoluble fibers.

A third aspect of the invention proposes a method for increasing the miscibility in aqueous liquid, the dispersibility in aqueous liquid, stability, and/or the bioavailability of bioactive and/or nutritional compounds of whole berries by using the process according to the first aspect of the invention.

A fourth aspect of the invention proposes an oral composition comprising the berry composition according to the second aspect of the invention, which is formulated as a food, a beverage, a food supplement, a pharmaceutical or cosmetic oral composition.

A fifth aspect of the invention proposes cosmetic use of a berry composition according to the second aspect of the invention or an oral composition according to the fourth aspect of the invention, for improving skin health in a healthy subject, in particular for photoprotection of the skin in a healthy subject or for protecting skin tissue against ageing in a healthy subject.

A sixth aspect of the invention proposes a berry composition according to the second aspect of the invention or an oral composition according to the fourth aspect of the invention, for use in a method for improving eye health.

A seventh aspect of the invention proposes a berry composition according to the second aspect of the invention or an oral composition according to the fourth aspect of the invention, for use in a method for boosting immunity.

An eighth aspect of the invention proposes a berry composition according to the second aspect of the invention or an oral composition according to the fourth aspect of the invention, for use in a method for preventing or treating cardiovascular diseases or disorders or cancers or diabetes.

These and other aspects, features and advantages of the invention will become more apparent to those skilled in the art from the detailed description of embodiments of the invention, in connection with the attached drawings.

### BRIEF DESCRIPTION OF THE DRAWINGS

FIG. 1 shows the amount of the content of dietary fibers (g/100 g) measured for compositions prepared with reference processes, i.e. milk/wolfberry compositions A and B, and a composition prepared with the process according to the invention, i.e. milk/wolfberry composition C.

FIG. 2 shows the retention rate of dietary fibers (%) measured for compositions prepared with reference processes, i.e. milk/wolfberry compositions A and B, and a composition prepared with the process according to the invention, i.e. milk/wolfberry composition C.

FIG. 3 shows the amount of the content of Vitamin C analog, in particular AA-2βG (mg/g) measured for compositions prepared with reference processes, i.e. milk/wolfberry compositions A and B, and a composition prepared with the process according to the invention, i.e. milk/wolfberry composition C.

FIG. 4 shows the retention rate of Vitamin C analog, in particular AA-2βG (%) measured for compositions prepared with reference processes, i.e. milk/wolfberry compositions A and B, and a composition prepared with the process according to the invention, i.e. milk/wolfberry composition C.

FIG. 5 shows the amount of the content of dipalmityl zeaxanthin (mg/g) measured for compositions prepared with reference processes, i.e. milk/wolfberry compositions A and B, and a composition prepared with the process according to the invention, i.e. milk/wolfberry composition C.

FIG. 6 shows the retention rate of dipalmityl zeaxanthin (%) measured for compositions prepared with reference processes, i.e. milk/wolfberry compositions A and B, and a composition prepared with the process according to the invention, i.e. milk/wolfberry composition C.

### DETAILED DESCRIPTION OF THE INVENTION

As used in the specification, the words “comprise”, “comprising” and the like are to be construed in an inclusive sense, that is to say, in the sense of “including, but not limited to”, as opposed to an exclusive or exhaustive sense.

Moreover, all numerical ranges should be understood to include each whole integer within the range.

As used in the specification, the singular forms “a”, “an”, and “the” include plural referents unless the context clearly dictates otherwise.

Unless noted otherwise, all percentages in the specification refer to weight percent, where applicable.

Unless defined otherwise, all technical and scientific terms have and should be given the same meaning as commonly understood by one of ordinary skill in the art to which this invention belongs.

As used in the present invention, a “berry” is intended to refer to, but is not limited to, fleshy fruit that typically has seeds, including any small, fleshy fruit popularly called a berry, especially if it is edible (e.g., wolfberry, cranberry, grape and the like). In particular, the term “berry” also refers to aggregate fruits developing from several ovaries, which are not berries in the botanical sense, but which are commonly accepted as “berry”. Exemplary suitable berries, also known as aggregate fruits, include raspberries, blackberries, strawberries and the like.

As used in the present invention, the term “skin” refers to the outermost envelope which is formed around the fruit and which is in direct contact with the air in the atmosphere. In particular, it corresponds generally to the outermost layer of the pericarp of the berry, which is called epicarp or exocarp.

As used in the present invention, the term “seeds” refers to the non-fleshy solid parts of a berry from which generally a new plant may grow. It also includes the achenes that could be found in some berries, e.g. strawberry.

As used in the present invention, the term “pulp” refers to the fleshy part of a berry, excluding the skin and the seeds. It may comprise large fibrous materials which are insoluble in water.

As used in the present invention, the term “bioactive and/or nutritional compound” is understood to mean molecules or components showing biological activity, health impact and/or participating in meeting the nutritional requirements of a subject when orally ingested.

As used in the present invention, the term “insoluble fibers” correspond to polysaccharides which do not dissolve into aqueous liquid, in particular water. It includes, for example, dietary fibers, in particular insoluble dietary fibers and starch.

As used in the present invention, the term “dietary fibers” refers to fibers that are not completely digestible by enzymes in the human gut system. Examples of dietary fibers include cellulose, hemicellulose, pectin, beta-glucans, gums, mucilages and lignin.

As used in the present invention, the term “insoluble dietary fibers” refers to dietary fibers which do not dissolve in water.

As used in the present invention, the term “soluble dietary fibers” refers to dietary fibers which dissolve in water.

As used in the present invention, the term “plant-based milk alternative” is a food product free from milk-derived components, consisting of one or several plant ingredients and mimicking the appearance, the texture and preferably, the nutritional properties of milk. In particular, it comprises plant proteins.

As used in the present invention, the whole berries: liquid medium ratio is a weight ratio.

As used in the present invention, D(90) particle size is a maximum particle size. In particular, 90% of the particles within a material, for example a food material, has a particle size less than D(90) particle size.

As used in the present invention, D(50) particle size is a maximum particle size. In particular, 50% of the particles within a material, for example a food material, has a particle size less than D(50) particle size.

As used in the present invention, D(10) particle size is a maximum particle size. In particular, 10% of the particles within a material, for example a food material, has a particle size less than D(10) particle size.

The particle sizes within the present application may be measured by means of laser diffraction technologies, e.g. using commercial instruments such as Malvern Mastersizer 3000 (Malvern Instruments, Malvern, UK).

In a first aspect, the invention relates to a process for preparing a berry composition. The berry composition may be in a liquid, powder or gel form.

The process comprises a step providing whole berries. Preferably, the whole berries are selected among whole wolfberries, whole blueberries, whole cranberries, whole white currants, whole red currants, whole blackcurrants, whole mulberries, whole blackberries, whole gooseberries, whole raspberries, whole sea buckthorns, whole strawberries, whole arbutus berries, whole grapes, or combinations thereof. More preferably, the whole berries comprise whole wolfberries. Most preferably, the whole berries consist only of whole wolfberries. Wolfberries are preferred as they are rich in bioactive and/or nutritional compounds and have many health-related, cosmetic and/or nutritional benefits, including benefits in relation with skin, eyes, immunity, heart, cancer and diabetes. The process is able to provide a berry composition which retain good properties, including good sensory and miscibility/dispersibility, even when only using whole wolfberries which have intricate botanical structure, including important fraction of insoluble compounds.

The whole berries are integral, i.e. the berries retain the essential overall structure and organization that they have just after having been harvested. In particular, they retain the essential botanical structures (e.g. skin, seeds, pulp etc. . . . ) that they have just after having been harvested. Particularly, the whole berries comprise skin, seeds and/or pulp and the skin, seeds and/or pulp are not removed from the whole berries.

The whole berries comprise skin, seeds and/or pulp which are intact. By “intact”, it is understood that the integrity of the skin, seeds and/or pulp of the berries has not been substantially affected by any mechanical, chemical or enzymatic processing. In other words, the skin, seeds and/or pulp has not been processed such that a portion of them has been removed from the berries or such that they have been transformed into a liquid, a powder and/or a suspension. For example, the skin, seeds and/or pulp of the berries has not been processed by cutting, grinding, milling, crushing, homogenisation, peeling, and/or hydrolysis.

In an embodiment, the water composition of the whole berries may have been changed, in particular decreased, compared to the water composition that the whole berries have just after being harvested, as long as they retain the skin, seeds and/or pulp of the whole berries. In particular, the whole berries may be dehydrated. The dehydration enables to decrease the water content in the berries and to concentrate the different compounds comprised in the berries, including their bioactive and/or nutritional compounds.

In an embodiment, the whole berries are not dehydrated or dried before step iii) of micronization. In particular, the whole berries are not dried to remove substantially all, preferably all of the water from the whole berries.

In a preferred embodiment, the whole berries are not frozen before step iii) of micronization. The freezing of the berries may negatively impact the structure of the berries and may destabilize, degrade or alter nutritional and bioactive compounds of the berries.

The whole berries are integral and comprises skin, seeds and/or pulp which are intact. Hence, the whole berries comprise inherent insoluble fibers, i.e. insoluble fibers which are inherent to the whole berries. The term “inherent insoluble fibers” excludes the exogenous insoluble fibers which are not inherently present in the whole berries and that could be added to the whole berries or any composition derived thereof, e.g. the berry suspension, the micronized berry suspension, the berry composition etc. Preferably, the inherent insoluble fibers are inherent insoluble dietary fibers. The whole berries are free from any exogenous insoluble fibers, in particular exogenous insoluble dietary fibers. The process does not comprise any step of addition of any exogenous insoluble fibers, in particular exogenous insoluble dietary fibers. Hence, the resulting berry composition is free from any exogenous insoluble fibers, in particular exogenous insoluble dietary fibers.

In an additional embodiment, the whole berries comprise inherent soluble fibers, i.e. soluble fibers which are inherent to the whole berries. The term “inherent soluble fibers” excludes the exogenous soluble fibers which are not inherently present in the whole berries and that could be added to the whole berries or any composition derived therefrom, e.g. the berry suspension, the micronized berry suspension, the berry composition etc. Preferably, the inherent soluble fibers are inherent soluble dietary fibers. The whole berries are free from any exogenous soluble fibers, in particular exogenous soluble dietary fibers. The process does not comprise any step of addition of any exogenous soluble fibers, in particular exogenous soluble dietary fibers. Hence, the resulting berry composition is free from any exogenous soluble fibers, in particular exogeous soluble dietary fibers.

The process further comprises a step of grinding the whole berries to prepare a berry suspension. For sake of clarity, this grinding step is not a micronization step. In particular the berry suspension has a D(90) particle size which is of at least 250 μm, preferably of 250 μm to 600 μm, more preferably of 300 μm to 500 μm, most preferably of 300 μm. For example, the grinding step may be performed by means of common techniques for mechanical comminution of food materials, in particular by means of general miller or colloid miller. An example of colloid miller is JM-L80 supplied by Wenzhou Longwan Huawei. This step enables a better performance of the micronization step. In particular, they allow rough milling/grinding of the whole berries to preliminary reduce their particle size before micronization and may also allow emulsification of the berry suspension. This grinding step may be performed in the presence of a liquid medium or in the absence of a liquid medium. If it is performed in the presence of a liquid medium, the liquid medium is preferably water, milk or milk protein-containing liquid medium.

Where the liquid medium is milk or milk-protein containing medium, the whole wolfberries are grinded in said milk or milk protein-containing liquid medium in a respective ratio of about 1:1 to 1:100, preferably from 1:1 to 1:50, more preferably 1:1 to 1:20, even more preferably 1:1 to 1:10, most preferably 1:5 to 1:10. The grinding step may be carried out at a temperature of from 1 to 95° C., preferably from about 20 to 80° C. and more preferably from 40 to 80° C.

The process further comprises a step of micronizing the whole berries or the berry suspension to prepare a micronized berry suspension. The micronization is a mechanical and high shearing operation to downsize the particles of food material to the micron range. In the present context, the term “micronizing the whole berries” or “micronizing the berry suspension” stands for grinding the whole berries or the berry suspension to achieve a micronized berry suspension having D(90) particle size of less than 200 μm, preferably less than 175 μm, more preferably less than 150 μm, even more preferably less than 100 μm. In an embodiment, the micronization results in a micronized berry suspension, and therefore in a berry composition, that has a D(10) particle size of less than 25 μm, preferably of 1 to 25 μm, a D(50) particle size of less than 50 μm, preferably of 30 μm to 50 μm and/or a D(90) particle size of less than 175 μm, preferably of 70 μm to 175 μm, more preferably of 70 μm to 100 μm.

The micronization makes possible the preparation of a berry composition that retains the majority of the bioactive and/or nutritional compounds comprised in whole berries, including the bioactive and/or nutritional compounds comprised in the solid and/or insoluble materials, generally coming from the skin, pulp and seeds which and are traditionally discarded/removed in the art. In particular, this makes possible the provision of a berry composition with enhanced nutritional properties. Indeed, the berry composition comprises a substantial amount of not only soluble fibers but also insoluble fibers which are traditionally discarded/removed. Thanks to the micronization step, the process generates limited or no waste and the extraction yield is significatively improved as the insoluble and/or solid materials, including insoluble fibers, are not discarded/removed. In particular, at least 90%, preferably at least 95% of all the compounds, including the different bioactive and/or nutritional compounds, from the whole berries used to prepare the berry composition are retained. Preferably, the bioactive and/or nutritional compounds in the berry composition obtained with the process of the invention have improved stability and/or bioavailability.

This improvement of the extraction yield is achieved without compromising the sensory and the miscibility/dispersibility properties of the berry composition. With micronization, whole berries with even their insoluble and/or solid materials are converted into a berry composition which has improved sensory, miscibility/dispersibility into aqueous medium, e.g. water. In particular, the composition has good miscibility/dispersibility into aqueous medium such as water, has homogenous texture and does not exhibit gritty/grainy texture even if the insoluble and/or solid materials are not discarded along the process.

The process provides a berry composition that has a profile of bioactive and/or nutritional compounds which is close to that naturally occurred in the whole berries. The process provides a berry composition with bioactive and/or nutritional compounds of whole berries, preferably in a highly bioavailable, stable and/or miscible form.

In an embodiment, the whole berries or the berry suspension may be micronized in presence of a medium, in particular in the presence of a liquid medium. The liquid medium may be plant-based milk alternative, milk, milk protein-containing liquid medium and/or water. The plant-based milk alternative is preferably soy milk. Preferably, the liquid medium may be milk, milk protein-containing liquid medium and/or water. Advantageously, the whole berries or the berry suspension are micronized in presence of a liquid medium and the liquid medium is milk or milk protein-containing liquid medium. Indeed, without wishing to be bound by theory, it is believed that the milk compounds, including milk proteins, further improve the properties of the berry composition, including g sensory and miscibility/dispersibility properties. It is also believed that the milk compounds further improve the bioavailability and the stability of the bioactive and/or nutritional compounds of the berry composition. Moreover, this may simplify the process by reducing the number of steps. Indeed, in that case, a further step of mixing and/or milling the micronized berry suspension in milk or milk protein-containing liquid medium is not needed.

When the liquid medium is milk or milk protein-containing liquid medium, the micronized berry suspension which results from the micronization step is a milk protein-containing micronized berry suspension.

In a preferred embodiment, the whole berries: liquid medium ratio during the micronization step may be of 1:1 to 1:100, preferably of 1:1 to 1:50, more preferably of 1:1 to 1:20, most preferably of 1:1 to 1:10.

In a further embodiment, the whole berries or the berry suspension may be micronized by performing 1 to 5 passes, preferably 1 to 3 passes into a micronization device or may be micronized for a time of at least 10 minutes, preferably for a time of 10 minutes to 900 minutes, more preferably 30 minutes to 200 minutes. This ensures sufficient micronization processing to achieve a berry composition with satisfactory sensory, stability and miscibility/dispersibility properties while minimizing the processing time.

In an embodiment, the micronization step may be carried out at a temperature of from 1 to 95° C., preferably from about 20 to 80° C. and more preferably from 40 to 80° C.

In an embodiment, the micronization step may be performed by means of high-pressure jet milling, by ball milling or by airflow impact grinding, preferably by means of high-pressure jet milling or by ball milling.

As mentioned above, the micronization may be performed by means of high-pressure jet milling. In this embodiment, the micronized berry suspension and the obtained berry composition may have a D(90) particle size of less than 100 μm, more preferably less than 95 μm. In particular, the micronized berry suspension and the obtained berry composition may have a D(10) particle size of less than 20 μm, preferably of 10 to 20 μm, a D(50) particle size of less than 45 μm, preferably of 35 μm to 45 μm and/or a D(90) particle size of less than 100 μm, preferably of 75 μm to 100 μm, more preferably 75 μm to 95 μm.

High-pressure jet milling is a milling technology that uses high density energy of cavitation and a high-pressure fluid jet to emulsify, homogenize and reduce the size of the particles of a material, such as a food material. In particular, a material containing solid particle is flowed into a channel, in particular a micro-channel and cavitation effects the ejection of a high-pressure fluid jet that impacts the material flow within the micro-channel. In particular, the cavitation makes the particles within the material impact, shock and collide, and this results to the reduction of the size of particles in the range of microns. An example of high-pressure jet milling equipment may be SPJ-500 made available by Beijing Institute of Collaborative Innovation (BICI), China.

Due to the very high flow rate of high-pressure jet milling, in particular between 300-450 m/s, the residence time of the material to be micronized in the channel of high-pressure jet milling device is very short, in particular less than 1 s. The micronization step may be achieved by performing 1 to 5, preferably 1 to 3 passes into a high-pressure jet milling device, preferably with a fluid jet pressure of 70 to 140 MPa, more preferably 100 to 140 MPa. More preferably, the micronization step may be achieved by performing 1, 2 or 3 passes, preferably with a fluid jet pressure of 70 to 140 MPa, more preferably 100 to 140 MPa, into a high-pressure jet milling device. Moreover, where the micronization step is performed by high-pressure jet milling, the micronization step is made in presence of a liquid medium as mentioned above. Preferably, the whole berries: liquid medium ratio during the micronization step with high-pressure jet milling may be of 1:1 to 1:20, preferably 1:2 to 1:10.

In an embodiment, the micronization step by means of high-pressure jet milling is performed with a berry suspension flow rate of 300-450 m/s. In a further embodiment, the micronization step by means of high-pressure jet milling may be carried out at a temperature of from 1 to 95° C., preferably from about 20 to 80° C. and more preferably from 40 to 80° C.

The different abovementioned high-pressure jet milling parameters participate in achieving an optimal size reduction of whole berries, including their insoluble and/or solid materials, into fine particles in the range of microns. This participates in achieving a berry composition with satisfactory and improved properties, in particular sensory and miscibility/dispersibility properties. Moreover, the bioactive and/or nutritional compounds in the berry composition may have improved bioavailability and/or stability.

Alternatively, the micronization may be performed by means of ball milling. In this embodiment, the micronized berry suspension and the obtained berry composition may have D(90) particle of less than 175 μm. In particular, the micronized berry suspension and the obtained berry composition may have a D(10) particle size of less than 15 μm, preferably of 5 to 15 μm, a D(50) particle size of less than 45 μm, preferably of 35 μm to 45 μm and/or a D(90) particle size of less than 175 μm, preferably of 135 μm to 175 μm.

Ball milling is a mechanical milling technology using a hollow cylindrical shell rotating around its axis, which is partially filled with balls made of a predetermined material e.g. steel, stainless steel, ceramic, rubber. Ball milling relies on the energy released from impact and attrition between the balls (grinding or milling medium) and the material to be grinded to reduce the particle size of the material to be grinded, in particular in the range of microns. Example of ball milling equipment include Nova S or CompactMix from Bulher or food high energy medium miller made available by Beijing Institute of Collaborative Innovation (BICI), China

When the micronization step is performed by means of ball milling, the whole berries may be micronized for a time of at least 10 minutes, preferably for a time of 10 minutes to 900 minutes. Preferably, the whole berries may be micronized for a time of 30 minutes to 600 minutes, more preferably of 30 minutes to 200 minutes, even more preferably of 60 minutes to 200 minutes, most preferably of 100 minutes to 170 minutes. In addition, the micronization step with ball milling is performed with rotational speed of 200 to 45000 rpm, preferably of 200 to 10000 rpm, more preferably of 200 to 5000 rpm. When the micronization step is performed by means of ball milling, the micronization step may be performed in the presence or the absence of a liquid medium as mentioned above. In particular, if the micronization step is made in presence of a hydrophilic liquid, the whole berries: hydrophilic liquid ratio during the micronization step with ball milling may be of 1:1 to 1:20, preferably of 1:1 to 1:10, more preferably 1:1 to 1:5, most preferably of 1:1 to 1:3.

In an embodiment, the micronization step by means of ball milling may be carried out with balls made of ceramic, preferably zirconium dioxide. Preferably, the balls may have a diameter of 0.5 mm to 100 mm, preferably of 0.5 mm to 50 mm, more preferably of 0.5 to 10 mm, even more preferably of 0.5 to 5 mm, even more preferably of 0.5 to 1.5 mm, most preferably of 0.7 mm.

In an embodiment, the micronization step by means of ball milling may be carried out at a temperature of from 1 to 95° C., preferably from about 20 to 80° C. and more preferably from 40 to 80° C.

The different abovementioned ball milling parameters participates in achieving an optimal size reduction of whole berries, including their insoluble and/or solid materials, into fine particles in the range of microns. This participates in achieving a berry composition with satisfactory and improved properties, including sensory and miscibility/dispersibility properties. Moreover, the bioactive and/or nutritional compounds in the berry composition may have improved bioavailability and/or stability.

Compared to ball milling, high-pressure jet milling allows a lower D(90) particle size while simplifying the process, in particular by minimizing the processing time. Hence, the micronization step is preferably performed by means of high-pressure jet milling.

In another alternative embodiment, the micronization step may be performed by airflow impact grinding. In this embodiment, the micronization step may be performed with a grind air volume of 30-1200 m³/h, preferably of 150 to 1000 m³/h and a pressure of 0.2-2.0 MPa, preferably of 0.8 to 1.5 MPa.

In an embodiment, the process does not comprise any step of addition of enzyme in the whole berries, in the berry suspension or in the micronized berry suspension before or during step iii) of micronization. In particular, the process does not comprise any step of addition of degrading enzymes in the whole berries, in the berry suspension or in the micronized berry suspension before or during step iii) of micronization. By “degrading enzyme”, it is understood an enzyme that degrades a molecule. For example, the degrading enzyme may be protein-degrading enzyme, fiber-degrading enzyme, polyphenol-degrading enzyme, carbohydrate-degrading enzyme or a mixture thereof. Examples of protein-degrading enzyme include protease, deamidase, proteinase or a mixture thereof. Examples of fiber-degrading enzyme include pectinase, cellulase, amylases, glucanase, xylanase or a mixture thereof. Examples of carbohydrate degrading enzyme include galactosidase, glucosidase, mannanase or a mixture thereof. Examples of polyphenol-degrading enzyme include tannase, carboxylic-ester hydrolase, naringinase, rhamnosidase or a mixture thereof. The addition of enzyme are not advantageous for the present invention because they may change negatively the nutritional content of the berries by degrading some key nutritional or bioactive compounds in the whole berries. For example, they may reduce the content of fibers, which are key nutritional compounds. In particular, a homogenous berry composition is obtained with the process of the invention without using any enzyme, in particular to degrade the insoluble compounds.

The process may further comprise an optional step of mixing and/or milling the micronized berry suspension in milk or milk protein-containing liquid medium to obtain a milk protein-containing micronized berry suspension. This step is not required if the micronization step is performed in presence of a medium which is milk or milk protein-containing liquid medium. The milk may be a skimmed milk, semi-skimmed milk and/or whole milk. In a preferred embodiment, the milk is non-human mammal milk, preferably cow milk. The milk protein-containing liquid medium may be any edible liquid containing milk proteins such as caseins or whey proteins, for example. Vegetable oils may optionally be added to the liquid medium. Preferably, this step is not optional.

The micronized berry suspension as described above, may be mixed and milled in said milk or milk protein-containing liquid medium in a respective ratio of about 1:1 to 1:100, preferably from 1:1 to 1:50, more preferably 1:1 to 1:20, even more preferably 1:1 to 1:10, most preferably 1:2 to 1:10. The mixing and/or milling step may be carried out at a temperature of from 1 to 95° C., preferably from about 20 to 80° C. and more preferably from 40 to 80° C.

Without wishing to be bound by theory, it is believed that the milk compounds, including milk proteins, further improve the properties of the berry composition, including sensory and miscibility/dispersibility properties and also further improve the bioavailability and the stability of the bioactive and/or nutritional compounds of the berry composition.

The process may further comprise an optional step of pasteurizing the micronized berry suspension or the micronized protein-containing berry suspension by techniques known in the art. For example, the micronized berry suspension or the micronized milk protein-containing berry suspension may be pasteurized by applying a heat-treatment at 75 to 90° C. for 10 to 60 min. In an embodiment, this pasteurization step is not optional.

The process may further comprise an optional step of adding synthetic or natural bioactive ingredients to the micronized berry suspension or the milk protein-containing micronized berry suspension. Examples of synthetic or natural bioactive ingredients include amino acids, fatty acids, vitamins, minerals, carotenoids, polyphenols, etc. They can be added to the micronized berry suspension or the milk protein-containing micronized berry suspension composition before the pasteurization step and/or drying step. They may be added by dry or by wet mixing to the micronized berry suspension or the milk protein-containing micronized berry suspension. In an embodiment, this addition step is not optional.

The berry composition may be used directly or concentrated or dried into powder for several applications into daily-consumed food products or other nutritional uses.

In particular, the process may further comprise an optional step of drying the micronized berry suspension or the milk protein-containing micronized berry suspension into a powder by techniques known in the art. For example, the drying step may be performed by spray drying or freeze drying. In an embodiment, this drying step is not optional.

In an embodiment, the process may further comprise an optional step of concentrating the micronized berry suspension or the milk protein-containing micronized berry suspension by techniques known in the art. For example, the concentration step may be performed by evaporation. In an additional embodiment, this concentration step is not optional.

The insoluble and/or solid materials, including inherent insoluble fibers are traditionally discarded in the art as they are not soluble and they negatively impact the properties of the berry composition, such as its sensory, miscibility/dispersibility properties. The process, including its micronization step, allows to overcome these drawbacks while bypassing any step of removal of insoluble and/or solid materials.

In particular, the process does not comprise any step of removal of the inherent insoluble fibers, preferably inherent insoluble dietary fibers coming from the whole berries. In particular, the process does not comprise any step of centrifugation and/or filtration and/or decantation to remove such inherent insoluble fibers, preferably inherent insoluble dietary fibers of the whole berries. In a further embodiment, the process does not comprise any step of removal of the insoluble and/or solid materials coming from the skin, the seeds and/or the pulp of the whole berries. In particular, the process does not comprise any step of centrifugation and/or filtration and/or decantation to remove such insoluble and/or solid materials coming from the skin, the seeds and/or the pulp of the whole berries.

In particular, thanks to the process of the invention, it is possible to achieve a berry composition with satisfactory and improved sensory and miscibility/dispersibility properties while retaining the bioactive and/or nutritional compounds, including the ones which are comprised in the solid and/or insoluble materials, generally coming from the skin, pulp and seeds. In particular, the process is simplified as steps such as centrifugation, filtration and/or decantation are not needed anymore to avoid compromising the properties of the final berry composition.

The process has the major advantage of being natural and cost effective enabling improved delivery of multi-nutrients, in particular bioactive and/or nutritional compounds from whole berries in the form of a combination of stabilized water- and fat-soluble compounds, free of organic solvent residues.

In a particular embodiment, the process does not involve the use of any organic solvents.

In a second aspect of the invention, the invention relates to a berry composition, in particular prepared with whole berries as described above. The berry composition may be in a liquid, powder or gel form.

The berry composition of the invention has a profile of bioactive and/or nutritional compounds which is close to that naturally occurred in whole berries. A high rate of bioactive and/or nutritional compounds is retained. The berry composition a substantial amount of soluble and insoluble fibers, in particular inherent soluble and insoluble fibers. Preferably, the berry composition of the invention has bioactive and/or nutritional compounds of berries preferably in a highly bioavailable, stable and/or miscible form.

The berry composition comprises lipophilic and hydrophilic, bioactive and/or nutritional compounds of a whole berry. Preferably, the berry composition comprises essentially all, more preferably all the lipophilic and hydrophilic, bioactive and/or nutritional compounds of a whole berry.

In preferred embodiment, the berry composition the berry composition retains at least 90%, preferably 95% of the content of essentially all or all lipophilic and hydrophilic, bioactive and/or nutritional compounds of a whole berry, in particular of the whole berries used to prepare the berry composition.

The lipophilic and hydrophilic, bioactive and/or nutritional compounds of a whole berry may comprise lipids, alkaloids, proteins, carbohydrates, carotenoids, polyphenolic compounds such as flavonoids, and vitamins or minerals, for example. In particular, the bioactive compounds may be flavonoids such as flavones (e.g. apigenin, luteolin or diosmetin), flavonols (e.g. quercetin, myricetin, kaempferol), flavanones (e.g. naringenin, hesperidin), catechins (e.g. epicatechin, gallocatechin), anthocyanidins (e.g. pelargonidin, malvidin, cyanidin) or isoflavones (e.g. genistein, daidzein); carotenoids such as carotenes and xanthophylls (e.g. lycopene, carotene, phytofluene, phytoene, canthaxanthin, astaxanthin, beta-cryptoxanthin, capsanthin, lutein, zeaxanthin, or those in the form of fatty acid esters; carbohydrates such as arabinogalactan proteins (e.g. lycium barbamm polysaccharide); vitamins (e.g. vitamin C, B, E . . . ); minerals (e.g. selenium, calcium, magnesium, potassium).

The berry composition may comprise a substantial amount of dipalmityl zeaxanthin which is key bioactive compounds of berries, in particular wolfberries. It is known to have several nutritional and health benefits. For example, it is known for having antioxidant properties and for contributing to eye health improvement. The amount of dipalmityl zeaxanthin in the berry composition may depend on the type of berries used, the amount of berries used and the whole berries: liquid medium ratio. In an embodiment, the berry composition may comprise at least 0.40 mg, preferably 0.40 to 0.80 mg, more preferably 0.40 to 0.50 mg, most preferably 0.40 to 0.45 mg of dipalmityl zeaxanthin per g of berry composition. For example, it may comprise 0.43 mg of dipalmityl zeaxanthin per g of berry composition.

The berry composition may comprise a substantial amount of vitamin C analog, in particular 2-O-(β-D-Glucopyranosyl) ascorbic acid which is also called AA-2βG. Vitamin C analog. Vitamin C analog is known to have several nutritional and health benefits. For example, it is known for having antioxidant and anti-aging properties, for enhancing immunity and for improving microbiota health. The amount of Vitamin C analog in the berry composition may depend on the type of berries used, the amount of berries used and the whole berries: liquid medium ratio. In an embodiment, the berry composition may comprise at least 1.70 mg of vitamin C analogs per g of berry composition. Preferably, the berry composition may comprise 1.70 mg to 5.00 mg vitamin C analogs, preferably 1.70 mg to 3.00 mg Vitamin C analogs, more preferably 1.80 mg to 2.00 mg Vitamin C analogs per g of berry composition.

In an embodiment, the berry composition is free from exogenous lipophilic and hydrophilic, bioactive and/or nutritional compounds. In other words, it is free from exogenous lipophilic and hydrophilic, bioactive and/or nutritional compounds which do not come from a whole berry, in particular which do not come from the whole berries used to prepare the berry composition and which are added to the berry composition. For example, the berry composition is free from exogenous dipalmityl zeaxanthin and/or exogenous Vitamin C analog.

In an embodiment, the berry composition the berry composition retains at least 90%, preferably 95% of the content of dipalmityl zeaxanthin and/or vitamin C analog of a whole berry, in particular of the whole berries used to prepare the berry composition.

The berry composition comprises the soluble and insoluble fibers of a whole berry. In particular, it comprises a substantial amount of soluble and insoluble fibers, which are known to entail many nutritional and health benefits. In particular, it has at least 1.5 wt. % of soluble and insoluble fibers, preferably inherent soluble and insoluble fibers. Preferably, the berry composition may comprise 1.5 wt. % to 5 wt. %, more preferably 2.0 wt. % to 3.0 wt. % of soluble and insoluble fibers. For example, it comprises 2.67 wt. % soluble and insoluble fibers.

In a preferred embodiment, the above-mentioned content refers to the amount of soluble and insoluble dietary fibers. In another embodiment, the above-mentioned content refers to the amount of inherent soluble and insoluble fibers, preferably inherent soluble and insoluble dietary fibers.

In a particular embodiment, the berry composition retains at least 90%, preferably at least 95% of soluble and insoluble fibers which are present in a whole berry, in particular in the whole berries used to prepare the berry composition. Preferably, the soluble and insoluble fibers are soluble and insoluble dietary fibers. In another embodiment, the soluble and insoluble fibers are inherent soluble and insoluble fibers, preferably inherent soluble and insoluble dietary fibers.

In an embodiment, the berry composition is free from exogenous soluble fibers and/or insoluble fibers, in particular exogenous soluble dietary fibers and/or exogenous insoluble dietary fibers.

The berry composition has fine particle size and exhibits improved sensory and miscibility/dispersibility properties. In particular, the berry composition has D(90) particle size of less than 200 μm, preferably less than 175 μm, more preferably less than 150 μm, even more preferably less than 100 μm, most preferably less than 95 μm.

In an embodiment, the berry composition may have a D(10) particle size of less than 25 μm, preferably of 1 to 25 μm, a D(50) particle size of less than 50 μm, preferably of 30 μm to 50 μm and/or a D(90) particle size of less than 175 μm, preferably of 70 μm to 175 μm, more preferably of 70 μm to 175 μm

In another embodiment, the berry composition may have a D(10) particle size of less than 20 μm, preferably of 10 to 20 μm, a D(50) particle size of less than 45 μm, preferably of 35 μm to 45 μm and/or a D(90) particle size of less than 100 μm, preferably of 75 μm to 100 μm, more preferably 75 μm to 95 μm.

In another embodiment, the berry composition have a D(10) particle size of less than 15 μm, preferably of 5 to 15 μm, a D(50) particle size of less than 45 μm, preferably of 35 μm to 45 μm and/or a D(90) particle size of less than 175 μm, preferably of 135 μm to 175 μm.

In a further embodiment, the berry composition may further comprise milk or milk protein-containing carrier. The milk may be a skimmed milk, semi-skimmed milk and/or whole milk. In a preferred embodiment, the milk is non-human mammal milk, preferably cow milk. The milk protein-containing carrier may be derived from any edible liquid containing milk proteins such as caseins or whey proteins, for example. The carrier may optionally comprise vegetable oils. It may also comprise plant proteins, such as soy proteins.

Without wishing to be bound by theory, it is believed that the milk compounds, including milk proteins, further improve the properties of the berry composition, including sensory and miscibility/dispersibility properties. These compounds are also believed to further improve the bioavailability and the stability of the bioactive and/or nutritional compounds of the berry composition.

In an additional embodiment, the composition may comprise one or more of emulsifiers, stabilizers, antioxidants and other additives. Use is made of emulsifiers compatible in food, such as phospholipids, for example lecithin, polyoxyethylene sorbitan mono- or tristearate, monolaurate, monopalmitate, mono- or trioleate, a mono- or diglyceride. Use may also be made of any type of stabilizer that is known in food, in cosmetics or in pharmaceuticals. Use is made of any type of antioxidants that is known in food, in cosmetics or in pharmaceuticals. Use is made, as additives, of flavorings, colorants and any other additive known in food, in cosmetics or in pharmaceuticals. These emulsifiers, stabilizers, antioxidants and additives are added according to the final use of the primary composition. The composition may also contain synthetic or natural bioactive ingredients such as amino acids, fatty acids, vitamins, minerals, carotenoids, polyphenols, etc.

In a preferred embodiment, the berry composition may be obtained according to the process provided in the first aspect of the invention.

The features of the berry composition provided of the first aspect of the invention applies to the berry composition of the second aspect of the invention, and vice versa.

In a third aspect the invention relates to a method for increasing the miscibility in aqueous liquid, the dispersibility in aqueous liquid, stability, and/or the bioavailability of bioactive and/or nutritional compounds of whole berries by using the process according to the first aspect of the invention.

As mentioned previously, the process of the invention effectively increases the miscibility and the dispersibility in aqueous liquid of bioactive and/or nutritional compounds of whole berries. Preferably, it also increases the stability, and/or the bioavailability of bioactive and/or nutritional compounds of whole berries.

In an embodiment, the whole berries which are used in the method are whole berries as described in the first aspect of the invention.

In an additional embodiment, the aqueous liquid may be plant-based milk alternative, milk, milk protein-containing liquid medium and/or water.

In a fourth aspect, the invention relates to an oral composition comprising the berry composition according to the second aspect of the invention. The oral composition is formulated as a food, a beverage, a food supplement, a pharmaceutical or cosmetic oral composition.

The berry composition may be formulated in a food or a beverage. The food may be a nutritional complete formula, a dairy product, a plant-based dairy analogue, a fermented dairy product, a dietary supplement, a meal replacement, a nutritional bar, a confectionery, an infant formula, an infant nutritional product, a cereal product or a fermented cereal-based product, an ice-cream, a chocolate, a culinary product such as mayonnaise, tomato puree or salad dressings or a pet food. The beverage may be a chilled or shelf stable beverage, a mineral or purified water, a liquid drink, a soup, a powdered drink or coffee.

In this case, the berry composition, which is preferably in the form of a powder, can be dispersed in the above-mentioned foods or beverages so as to have a daily intake in bioactive and/or nutritional compounds as described in the second aspect of the invention, which depends mainly on the berry utilized, the desired effect and target tissue. The amount of the berry composition, food or beverage to be consumed by the individual to obtain a beneficial effect will also depend upon its size, its type, and its age.

The berry composition may be formulated in a food supplement. The food supplement may be in capsules, gelatin capsules, soft capsules, tablets, sugar-coated tablets, pills, pastes or pastilles, gums, or drinkable solutions or emulsions, a syrup or a gel, with a dose of about 0.1 to 100% of the berry composition, which can then be taken directly with water or by any other known means. This supplement may also include a sweetener, a stabilizer, an antioxidant, an additive, a flavoring or a colorant. A supplement for cosmetic purpose can additionally comprise a compound active with respect to the skin. Methods for preparing them are common knowledge.

The berry composition may be formulated in a pharmaceutical oral composition. The pharmaceutical oral composition can be administered for prophylactic and/or therapeutic treatments. In therapeutic applications, compositions according to the invention are administered to a patient already suffering from a disease, as described herein under, in an amount sufficient to cure or at least partially arrest the symptoms of the disease and its complications. An amount adequate to accomplish this is defined as “a therapeutically effective dose”. Amounts effective for this will depend on the severity of the disease and the weight and general state of the patient. In prophylactic applications, compositions according to the invention are administered to a patient susceptible to or otherwise at risk of a particular disease. Such an amount is defined to be “a prophylactic effective dose”. In this use, the precise amounts again depend on the patient's state of health and weight.

The pharmaceutical oral composition of the invention is preferably administered with a pharmaceutically acceptable carrier, the nature of the carrier being adapted for oral routes. The desired formulation can be made using a variety of excipients including, for example, pharmaceutical grades of mannitol, lactose, starch, magnesium stearate, sodium saccharin, cellulose, magnesium carbonate.

In a preferred embodiment, the pharmaceutical oral composition may be a tablet, a capsule, a pill, a solution, a suspension, a syrup, a dried oral supplement, a wet oral supplement.

It will be appreciated that the skilled person will, based on his own knowledge select the appropriate components and galenic form to target the active compound to the tissue of interest, e.g. the skin, colon, stomach, eyes, kidney or liver, taking into account that the route of administration is oral.

The invention also relates to a cosmetic oral composition comprising the berry composition described above. As the pharmaceutical oral composition, the cosmetic oral composition may be a tablet, a capsule, a pill, a solution, a suspension, a syrup, a dried oral supplement, a wet oral supplement. It is also possible to add other cosmetically active ingredients. Excipients or colorants commonly used in cosmetic can also be added to the composition.

It will be understood that the concept of the present invention may likewise be applied as an adjuvant therapy assisting in presently used medications. Since the berry composition of the present invention may easily be administered together with food material, special clinical food may be applied containing a high amount of the said berry composition. It will be clear that on reading the present specification together with the appending claims the skilled person will envisage a variety of different alternatives to the specific embodiments mentioned herein.

In a fifth aspect, the invention relates to the cosmetic use of a berry composition according to second aspect of the invention or an oral composition according to the fourth aspect of the invention, for improving skin health, in particular in a healthy subject. The subject may be a pet or a human. In particular, they may be used for photoprotection of the skin in a healthy subject or for protecting skin tissue against ageing in a healthy subject, e.g. for inhibiting damage to the skin and/or mucous membranes by inhibiting collagenases and enhancing the synthesis of collagen. The berry composition comprises bioactive and/or nutritional compounds which are beneficial for the skin. In particular, the use of the berry composition of the invention makes it possible to enhance the bioavailability in the body of said bioactive and/or nutritional compounds present originally in whole berries to improve skin health, e.g. to slow down the ageing of the skin. It may also be used for improving skin density or firmness.

In a sixth aspect, the invention relates to a berry composition according to second aspect of the invention or an oral composition according to the fourth aspect of the invention, for use in a method for improving eye health in a subject. The subject may be a pet or a human. Preferably, the subject is healthy. In particular, it may be used for reducing risk of cataract and age-related macular degeneration in a subject. The berry composition comprises bioactive and/or nutritional compounds which are beneficial for the eyes. In particular, the use of the berry composition as described above makes it possible to enhance the bioavailability in the body of said bioactive and/or nutritional compounds present originally in whole berries to improve eye health.

In a seventh aspect, the invention relates to a berry composition according to second aspect of the invention or an oral composition according to the fourth aspect of the invention, for use in a method for boosting immunity. This may be used in a method for boosting immunity in subject which may be a human or a pet. In particular, the berry composition according to second aspect of the invention or the oral composition according to the fourth aspect may be used in a method for stimulating an immune response to an antigen administered to a subject in need thereof. The subject may be a human or a pet. The subject may be healthy. In a preferred embodiment, the antigen is a vaccine for a disease or disorder afflicting said subject or to which said subject is susceptible. More preferably, the antigen is a vaccine for treating or preventing infections to coronavirus or influenza virus. For example, the influenza virus may be an influenza virus belonging to Influenza A, Influenza B or Influenza C genus. For example, the coronavirus may be HCoV-OC43, HCoV-HKU1, HCoV-229E, HCoV-NL63, SARS-COV, MERS-COV, or covid-19 (also named SARS-COV-2). Most preferably, the antigen is a vaccine for treating or preventing infections to covid-19. The berry composition comprises bioactive and/or nutritional compounds which are beneficial for boosting the immunity. In particular, the use of the berry composition as described above makes it possible to enhance the bioavailability in the body of said bioactive and/or nutritional compounds present originally in whole berries to boost immunity, including to stimulate the immune response in the frame of vaccination.

In an eighth aspect, the invention relates to a berry composition according to second aspect of the invention or an oral composition according to the fourth aspect of the invention, for use in a method for preventing or treating cardiovascular diseases or disorders or cancers or diabetes, in particular in a subject. The subject may be a human or a pet. The subject may be healthy. The berry composition comprises bioactive and/or nutritional compounds which are beneficial for preventing/treating cardiovascular diseases or disorders, cancers or diabetes. In particular, the use of the berry composition as described above makes it possible to enhance the bioavailability in the body of said bioactive and/or nutritional compounds present originally in whole berries to prevent/treat cardiovascular diseases or disorders, cancers or diabetes.

Those skilled in the art will understand that they can freely combine all features of the present invention disclosed herein. In particular, features described for the product of the present invention may be combined with the process, the method and the use(s) of the present invention and vice versa. Further, features described for different embodiments of the present invention may be combined.

Furthermore, where known equivalents exist to specific features, such equivalents are incorporated as if specifically referred in this specification. Further advantages and features of the present invention are apparent from the figures and non-limiting examples.

### EXAMPLES

### Example 1: Effect of the Grinding Technology on the Particle Size and the Properties of the Resulting Wolfberry Composition

Four wolfberry compositions were prepared by grinding whole wolfberries, including skin, seeds and pulps, with different grinding technologies.

A first wolfberry composition, hereinafter grinded wolfberry composition, was prepared by grinding the whole wolfberries with two passes in a colloid miller (JM-L80, Wenzhou Longwan Huawei, China) in presence of water. In particular, the whole wolfberries: water ratio is of 1:1.2.

A second wolfberry composition, hereinafter ball-milled wolfberry composition, was prepared by micronizing the first wolfberry composition with a ball miller (Food high energy medium miller, BICI, China) equipped with balls (zirconium dioxide, 0.7 mm in diameter) at a rotation speed 3000 rpm, in presence of water for 150 minutes. As for the first wolfberry composition, the whole wolfberries: water ratio is of 1:1.2.

A third wolfberry composition, hereinafter jet-milled wolfberry composition, was prepared by micronizing the first wolfberry composition in presence of water with 2 passes by high pressure jet miller (SPJ-500, BICI, China) with fluid jet pressure of 120 MPa. As for the first wolfberry composition, the whole wolfberries: water weight ratio is of 1:6.

The particle size of the different wolfberry compositions, including D(10), D(50) and D(90) particle sizes were assessed by laser diffraction (Malvern Mastersizer 3000, Fraunhofer algorithm, dispersion water)

The results are provided in Table 1.

TABLE 1

| | D(10) (μm) | D(50) (μm) | D(90) (μm) |
|---|---|---|---|
| Grinded wolfberry | 41.3 | 157 | 565 |
| composition | | | |
| Ball-milled wolfberry | 9.35 | 39.5 | 147 |
| composition | | | |
| Jet-milled wolfberry | 15.4 | 40.6 | 81.1 |
| composition | | | |

It is observed that the grinded wolfberry composition exhibits larger particle size and a wider particle size range compared to that of ball-milled wolfberry composition and jet-milled wolfberry composition. In opposition to the standard grinding technology, the ball milling and jet milling technologies are effective technologies to provide wolfberry compositions with fine particle size. Despite the presence of skin, seeds and pulp, including insoluble fibers, ball milling and jet milling technologies allow the provision of wolfberry compositions with substantially lower D(10), D(50) and D(90) particle size compared to the standard grinding technology (cf. Table 1).

In addition, it is observed that jet milling technology is advantageous over ball milling technology. Indeed, jet milling provides wolfberry compositions with narrower particle size range, in particular with a lower D(90) than ball milling. In particular, jet milling technology achieves low particle size in a lower time than ball milling, few seconds versus 150 minutes.

The different compositions were tasted. The ball-milled and jet-milled wolfberry compositions exhibit homogenous texture without grainy/gritty sensation in mouth and good taste. In opposition, the grinded wolfberry composition exhibits heterogenous texture with grainy/gritty sensation in mouth.

The different compositions were added into an aqueous liquid, i.e. water. The ball-milled and jet-milled wolfberry compositions exhibit good miscibility and dispersion properties and provide a homogenous solution. The grinded wolfberry compositions exhibit unsatisfactory miscibility and dispersion properties and provide a heterogenous solution with solid particles visible to the naked eyes.

Despite the use of whole wolfberries and the absence of removal step of insoluble and/or solid materials, including insoluble fibers, it is concluded that jet milling and ball milling technologies are effective to provide wolfberry compositions with improved sensory, miscibility and dispersibility properties.

### Example 2: Particle Size of Wolfberry Composition Prepared with a Method of the Prior Art

A wolfberry composition was prepared according a prior art process. This process does not involve micronization technologies, such as jet milling or ball milling. It involves simple grinding steps with colloid miller. In addition, the process comprises centrifugation and decanting step to remove solid and insoluble materials, such as insoluble fibers. Solid and/or insoluble materials, including insoluble fibers, are removed to avoid that they negatively impact the sensory and miscibility/dispersibility properties of the wolfberry.

In particular, the prior art wolfberry composition was prepared accordingly. Wolfberry fruits (4 kg) and reconstituted skimmed milk (16 L) were introduced in a 50-liter container. The mixture was kept to stand for 10 minutes and grinded with a colloid miller (JM50, Wenzhou Colloid Miller Factory, China) with 2 passes. The second pass in the colloid miller was performed with a minimal gap distance of 5 μm. The temperature of the mixture was maintained at 80-85° C. by means of water bath and cooled to room temperature afterwards. The resulting mixture was then centrifuged at 2000 G for 10 minutes. The solid residue, including insoluble fibers, is discarded.

The particle size of the prior art wolfberry composition, including D(10), D(50) and D(90) particle sizes were assessed with the method provided in Example 1 and were compared to the particle size values measured for ball-milled and jet-milled wolfberry composition prepared in Example 1.

The results are provided in table 2.

TABLE 2

| | D(10) (μm) | D(50) (μm) | D(90) (μm) |
|---|---|---|---|
| Prior art wolfberry | 11.8 | 32.3 | 72.1 |
| composition | | | |
| Ball-milled wolfberry | 9.35 | 39.5 | 147 |
| composition | | | |
| Jet-milled wolfberry | 15.4 | 40.6 | 81.1 |
| composition | | | |

It is observed that jet milling and ball milling provides wolfberry compositions with fine particle size as for the wolfberry composition obtained with the process of the prior art. For example, the D(50) particle size are similar for the three wolfberry compositions. However, jet milling and ball milling achieve fine particle size without using any step for removing solid and/or insoluble materials, including insoluble fibers. Hence, it can be concluded that the micronization technologies, in particular jet milling and ball milling, can realize the multi-effect of grinding, centrifugation and filtration. The micronization technologies allow to retain the insoluble and/or solid materials without impacting the sensory and miscibility/dispersibility properties of the wolfberry composition. This opens the way of improving the extraction yield. Indeed, the compounds of interest (e.g., bioactive compounds, nutritional compounds) that were excluded with insoluble and/or solid materials in the prior art are now retained without negative impact on wolfberry compositions properties.

### Example 3: Particle Size of Wolfberry Composition Prepared with Milk

Three wolfberry compositions were prepared by grinding or micronizing whole wolfberries, including skin, seeds and pulps, in presence of milk.

A first composition was prepared by grinding whole wolfberry composition and by mixing said composition with skimmed milk, without subsequent centrifugation steps, hereinafter milk/wolfberry composition A. It was prepared by mixing the first wolfberry composition obtained in example 1 using Silverson L4RT at 4000 rpm for 10 minutes in presence of skimmed milk. The insoluble and/or solid materials, including insoluble fibers, are not removed. In particular, the wolfberry composition: skimmed milk ratio is of 1:1.

A second composition, was prepared by grinding whole wolfberry composition and by mixing said composition with skimmed milk with subsequent centrifugation step, hereinafter milk/wolfberry composition B. It was prepared by mixing the first wolfberry composition obtained in example 1 using Silverson L4RT at 4000 rpm for 10 minutes in presence of skimmed milk to obtain a suspension. In particular, the wolfberry composition: skimmed milk ratio is of 1:1. The resulting suspension is thereafter centrifugated and the insoluble and/or solid residues, including insoluble fibers, are removed.

A third composition, was prepared by micronizing whole wolfberry composition and by mixing with skimmed milk without subsequent centrifugation steps, hereinafter milk/wolfberry composition C. It was prepared by mixing the third wolfberry composition obtained in example 1 using Silverson L4RT at 4000 rpm for 10 minutes in presence of skimmed milk. In particular, the whole wolfberry composition: skimmed milk ratio is of 1:1.

The particle size of the different compositions, including D(10), D(50) and D(90) particle sizes were assessed with the method of example 1.

The results are provided in table 3.

TABLE 3

| | D(10) (μm) | D(50) (μm) | D(90) (μm) |
|---|---|---|---|
| Milk/wolfberry | 18.5 | 71.3 | 349 |
| composition A | | | |
| Milk/wolfberry | 3.46 | 35.8 | 89.0 |
| composition B | | | |
| Milk/wolfberry | 8.05 | 41.1 | 160 |
| composition C | | | |

The results of Table 3 are consistent with the results provided in Example 2. In particular, it can be observed that micronization enables to achieve a wolfberry composition C with fine particle size without using any centrifugation and/or filtration step to remove insoluble and/or solid materials. In particular, the D(90) particle size of the milk/wolfberry composition of the milk/wolfberry composition C is substantially lower than for milk/wolfberry composition obtained by grinding without centrifugation, i.e. composition A. The D(90) particle size of the milk/wolfberry composition C is higher than the milk/wolfberry composition B but the overall particle size remains fine and satisfactory.

In particular, the different compositions were tasted. The milk/wolfberry compositions B and C exhibit homogenous texture without grainy/gritty sensation in mouth and good taste. In opposition, the milk/wolfberry composition A exhibits heterogenous texture with unpleasant grainy/gritty sensation in mouth.

Moreover, the different compositions were added into an aqueous liquid, i.e. water. The milk/wolfberry compositions B and C exhibit good miscibility and dispersion properties and provide a homogenous solution. The milk/wolfberry composition A exhibits unsatisfactory miscibility and dispersion properties and provide a heterogeneous solution with solid particles visible to the naked eyes.

Hence, the milk/wolfberry compositions B and C exhibit good sensory and miscibility/dispersibility properties. However, compositions obtained by simple grinding require centrifugation step and removal step of insoluble and/or solid materials to exhibit such satisfactory properties. On the contrary, compositions obtained by micronization do not require such steps and micronization is sufficient by itself to achieve such satisfactory properties. Hence, micronization allows to retain the insoluble and/or solid materials, including the bioactive and/or nutritional compounds of interest they contain, without compromising the sensory and miscibility/dispersibility properties of the final wolfberry composition. In particular, during centrifugation and removal step, a mass loss of 47.13% has been measured. This underlines that a substantial part of bioactive and/or nutritional compounds of interest are lost during these steps.

In addition, it is expected that milk/wolfberry composition C has enhanced stability, bioavailability of its bioactive and/or nutritional compounds.

### Example 4: Characterization of Key Bioactive and/or Nutritional Compounds in Wolfberry Compositions

The content and the retention rate of different key active bioactive and/or nutritional compounds of whole wolfberries were measured in the different compositions of example 3. In particular, the content and retention rate were assessed for the following compounds: dietary fibers, vitamin C analog (i.e. AA-2βG) and dipalmityl zeaxanthin.

The retention rate corresponds to the % of compounds from the whole berries used to prepare the milk/wolfberry compositions remain in the milk/wolfberry composition.

Dietary fibers were measured with method provided in AOAC 991.43. In particular, the samples underwent sequential enzymatic digestion by heat-stable alpha-amylase, protease and amyloglycosidase to remove starch and protein. Afterwards, the obtained enzyme digestate was treated with alcohol to precipitate soluble dietary before filtration. After filtration the total dietary fiber (TDF) residue was washed with alcohol and acetone, was dried and was weighed. TDF values were corrected for protein, ash.

Vitamin C analog was measured with the HPLC method published in Chinese Journal of Food Safety & Quality, 2021,12 (20). In particular, Vitamin C analog was extracted with water. Afterwards, it was separated on a poroshell 120 HILIC column, eluted by 90% acetonitrile (V: V) and 10% phosphoric acid water solution and then identified by ultraviolet (UV) detector at 235 nm and quantified.

Dipalmityl Zeaxanthin was measured by the HPLC method published in Chinese Journal of Food Safety & Quality 2021,12 (20). In particular, Dipalmityl Zeaxanthin was extracted with dichloromethane/ethanol (v: v, 8:2) and was separated on a Dionex Acclaim C30 column, eluted by methanol/methyl tert-butyl ether, identified by UV detector at 452 nm and quantified.

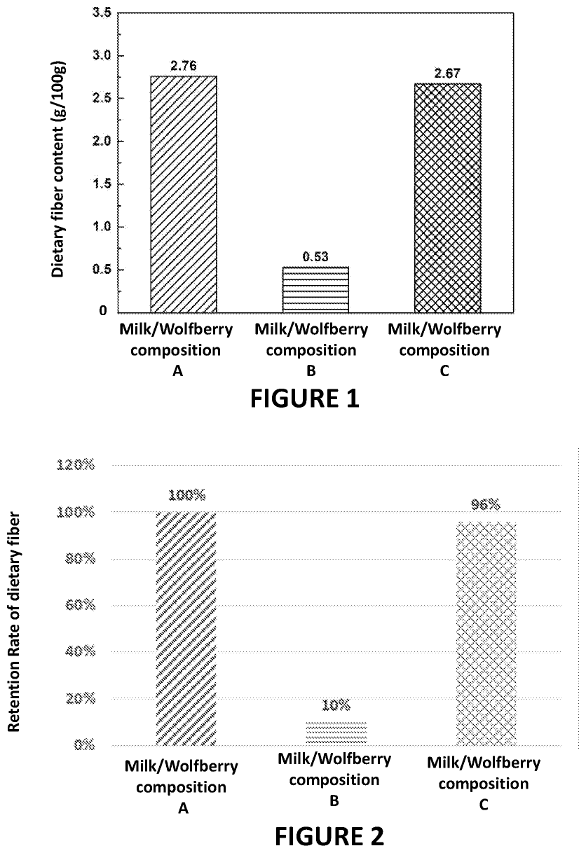

The results for dietary fibers are shown in FIGS. 1 and 2. On one side, it can be seen that milk/wolfberry compositions A and C exhibit high retention rate (>95%) of dietary fibers, and substantial amount of dietary fibers (>2.5 wt %). On the other side, it can be observed that the milk/wolfberry composition B has poor retention rate, i.e. only 10%, of dietary fibers and comprises an amount of only 0.53 wt. %. Hence, the centrifugation and removal steps lead to the loss of a major part of the dietary fibers, which are compounds of high nutritional/health value.

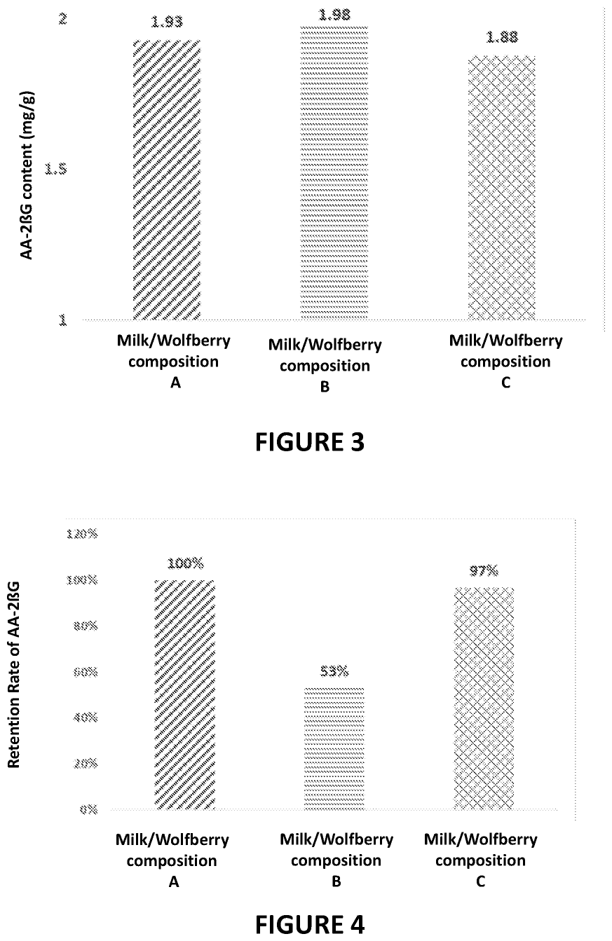

The results for AA-2βG are shown in FIGS. 3 and 4. On one side, it can be seen that milk/wolfberry compositions A and C exhibit high retention rate (>95%) of AA-2G, while milk/wolfberry composition C has low retention rate of only 53%. The amount in mg/g of Vitamin C analog are comparable between the three compositions as it was very water soluble and evenly distributed into whole matrix.

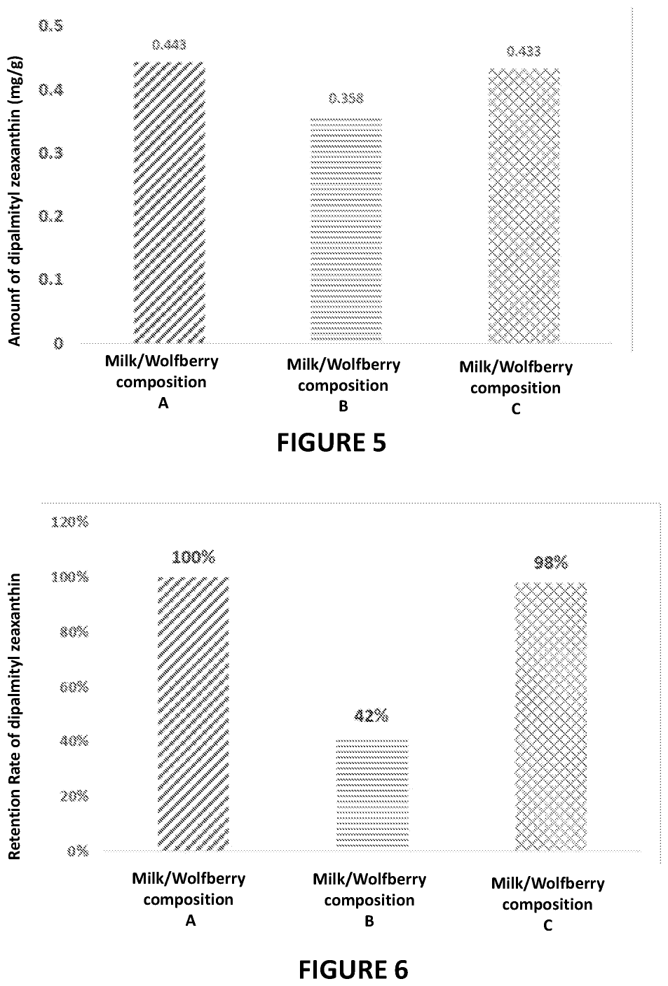

The results for dipalmityl zeaxanthin are shown in FIGS. 5 and 6. On one side, it can be seen that milk/wolfberry compositions A and C exhibit high retention rate above 95% of dipalmityl zeaxanthin, and substantial amount of dipalmityl zeaxanthin with around 0.43-0.44 mg/g. On the other side, it can be observed the milk/wolfberry composition B has low retention rate, i.e. only 42%, of dipalmityl zeaxanthin and comprises an amount of only 0.36 mg/g of dipalmityl zeaxanthin. Hence, the centrifugation and removal steps lead to the loss of a major part of the dipalmityl zeaxanthin, which is key bioactive and/or nutritional compounds from wolfberries.

As a conclusion, it can be concluded that centrifugation and removal steps lead to the loss of a substantial part of key bioactive and/or nutritional compounds comprised in the initial wolfberries. In particular, the processes without centrifugation (i.e. compositions A and C) exhibit the highest retention rate of the different compounds with substantially high amounts of respective bioactive and/or nutritional compounds. However, as provided in example 2, composition A has unsatisfactory sensory and miscibility/dispersibility properties. In other words, micronization (cf. composition C) is the most advantageous technology. Indeed, it provides at the same time 1) high retention rate of bioactive and/or nutritional compounds comprised in initial whole wolfberries and 2) wolfberry compositions with good sensory, miscibility/dispersibility properties.

### Example 5: Preparation of Wolfberry Composition by Micronizing with Milk

A jet-milled wolfberry composition was prepared with milk. In particular, the first wolfberry composition of example 1 was further micronized with three passes in a jet milling equipment (SPJ-500, BICI, China) in presence of milk with fluid jet pressure of 120 MPa. In particular, the whole wolfberries: skimmed milk weight ratio is of 1:6. After micronization, the temperature of the mixture was maintained at 80-85° C. by means of water bath and cooled to room temperature afterwards to obtain a final wolfberry composition.

It is expected to obtain a final wolfberry composition with good sensory, miscibility/dispersibility properties and with enhanced stability, bioavailability of its bioactive and/or nutritional compounds.

### Example 6: Preparation of Wolfberry Powder

The ball milled wolfberry composition of example 1, the jet-milled wolfberry composition of example 1 or the milk/wolfberry composition C of example 3 or the wolfberry composition of example 5 were prepared according to their respective processes as disclosed in example 1, 3 or 5. After their preparation, the wolfberry compositions were freeze-dried to obtain a wolfberry powder.

It is expected that the obtained wolfberry powder has good reconstitution properties into aqueous liquid such as milk or water. The obtained liquid wolfberry composition after reconstituting the powder into aqueous liquid is expected to have good sensory and miscibility/dispersibility properties.

### Example 7: Dairy Product Containing Wolfberry Composition According to the Invention

The ball-milled wolfberry composition of example 1, the jet-milled wolfberry composition of example 1, the milk/wolfberry composition C of example 3, the wolfberry composition of example 5, the wolfberry powder of example 6 was used for the manufacture of fermented yogurt-like milk products.

To do this, 1 L of a milk product containing 2.8% of fats and supplemented with 2% of skimmed milk powder and 6% of sucrose was prepared, pasteurized and its temperature then lowered to 42° C. Precultures of a non-thickening strain of Streptococcus thermophilus and of a non-viscous strain of Lactobacillus bulgaricus were reactivated in a sterile MSK culture medium containing 10% of reconstituted milk powder and 0.1% of commercial yeast extract. The pasteurized milk product is then inoculated with 1% of each of these reactivated precultures and this milk product was then allowed to ferment at 32° C. until the pH reached a value of 4.5. To the fermented milk, yogurt-like product, one of the above-mentioned wolfberry composition (1%) was added and stored at 4° C.

### Example 8: Pet Food Product

A feed mixture was made up of corn, corn gluten, chicken and fish, salts, vitamins and minerals. The moistened feed leaving the pre-conditioner was then fed into an extruder-cooker and gelatinised. The gelatinised matrix leaving the extruder was forced through a die and extruded. The extrudate leaving the die head was cut into pieces suitable for feeding to dogs, dried at about 110° C. for about 20 minutes, and cooled to form pellets. The resulting water activity of the pellets was about 0.6.

The pellets were coated by spraying a coating substrate comprising tallow fat and any one of the wolfberry compositions mentioned in example 8.

### Example 9: Cosmetic for Oral Administration

A composition in the form of a hard capsule has the following formulation (cf. table 4):

TABLE 4

| Compound | mg per capsule |
|---|---|
| Wolfberry composition | 500 |
| Excipient for the core | |
| Microcrystalline cellulose | 70 |
| Encompress TM | 60 |
| Magnesium stearate | 3 |
| Anhydrous colloidal silica | 1 |
| Coating agent | |
| Gum-lac | 5 |
| Talc | 61 |
| Sucrose | 250 |
| Polyvidone | 6 |
| Titanium dioxide | 0.3 |
| Coloring agent | 5 |

The wolfberry composition is any one of the wolfberry compositions mentioned in example 7.

The composition can be administered to the individual in an amount of 2 to 3 capsules daily.

### Example 10: Beverage

A beverage was prepared was by using any one of the following wolfberry compositions: the ball-milled wolfberry composition of example 1, the jet-milled wolfberry composition of example 1, the milk/wolfberry composition C of example 3, the wolfberry composition of example 5. In particular, the wolfberry composition was mixed with Vitamin E, phosphate buffer, complex stabilizer (including emulsifier, hydrocolloid) to obtain a mixture. Then, the resulting mixture underwent indirect UHT treatment (e.g. 135° C., 6 seconds) and aseptic filling in bottles to obtain a beverage.

### Example 11: Preparation of a Cranberry Composition

A cranberry composition was prepared with whole cranberries. In particular, the whole cranberries were micronized with two passes into a jet milling equipment with fluid jet pressure of 120 MPa in presence of whole milk. In particular, the whole cranberries: whole milk ratio was 1:12.

It is expected to obtain a final cranberry composition with good sensory, miscibility/dispersibility properties and with enhanced stability, bioavailability of its bioactive and/or nutritional compounds.

### Example 12: Preparation of a Strawberry Composition

A strawberry composition was prepared with whole strawberries. In particular, the whole strawberries were micronized with two passes into a jet milling equipment with a fluid jet pressure of 120 MPa in presence of skimmed milk. In particular, the whole strawberries: whole milk ratio was 1:6.

It is expected to obtain a final strawberry composition with good sensory, miscibility/dispersibility properties and with enhanced stability, bioavailability of its bioactive and/or nutritional compounds.

Although the invention has been described by way of example, it should be appreciated that variations and modifications may be made without departing from the scope of the invention as defined in the claims.

1. A process for preparing a berry composition comprising the steps consisting of:
i) providing whole berries, wherein the whole berries comprise skin, seeds and/or pulp which are intact, and wherein the whole berries comprise inherent insoluble fibers,
ii) grinding the whole berries to prepare a berry suspension, and
iii) micronizing said whole berries in presence of a liquid medium to prepare a micronized berry suspension having D(90) particle size of less than 200 μm,
wherein the process does not comprise any step of removal of the inherent insoluble fibers coming from the whole berries.

2. The process according to claim 1, wherein the whole berries are selected from the group consisting of whole wolfberries, whole blueberries, whole cranberries, whole white currants, whole red currants, whole blackcurrants, whole mulberries, whole blackberries, whole gooseberries, whole raspberries, whole sea buckthorns, whole strawberries, whole arbutus berries, whole grapes, and combinations thereof.

3. The process according to claim 2, wherein the whole berries consist only of whole wolfberries.

4. The process according to claim 1, wherein the whole berries: liquid medium ratio during the micronization step is of 1:1 to 1:20.

5. The process according to claim 1, wherein the whole berries are micronized by performing 1 to 5 passes into a micronization device.

6. The process according to claim 1, wherein the micronization step iii) is performed by means of high pressure jet milling, ball milling or airflow impact grinding.

7. A berry composition which comprises lipophilic and hydrophilic, bioactive and/or nutritional compounds of a whole berry, which comprises the soluble and insoluble fibers of a whole berry, which has a D(90) particle size of less than 200 μm and which has at least 1.5 wt. % of soluble and insoluble fibers.

8. The berry composition according to claim 7, which comprises at least 1.70 mg of vitamin C analogs per g of berry composition and/or at least 0.40 mg of dipalmityl zeaxanthin per g of berry composition.

9. The berry composition according to claim 7, wherein it further comprises milk or milk protein-containing carrier.

10-18. (canceled)
